(19) United States
(12) Patent Application Publication
(10) Pub. No.: US 2006/0247255 A1
(43) Pub. Date: Nov. 2, 2006

(54) Method for preparing a stable gatifloxacin composition

(76) Inventors: Satishkumar Ambalal Patel, West Windsor, NJ (US); Suresh Palaniswamy, East Windsor, NJ (US); Bhaskarbhai C. Patel, Edison, NJ (US); Pablo Davila, East Windsor, NJ (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/119,543

(22) Filed: May 2, 2005

(51) Int. Cl. A61K 31/496

(52) U.S. Cl. 514/253.08

(57) ABSTRACT

A method for preparing a stable gatifloxacin composition in oral dosage form, said method comprising: (a) mixing gatifloxacin or a pharmaceutically acceptable salt or hydrate thereof, a disintegrant, and a filler for a time sufficient to form a mixture; (b) compacting the mixture of Step (a) at a compaction force of about 1 kN to about 30 kN for a time sufficient to form a compact; (c) milling the compact of Step (b) to form a granulation; and (d) mixing the granulation of Step (c) with an effective amount of a lubricant for a time sufficient to produce a gatifloxacin composition having a tap density of at least about 0.50 g/mL.

### FIELD OF THE INVENTION

The present invention provides a method for preparing a stable gatifloxacin composition.

### BACKGROUND OF THE INVENTION

Gatifloxacin is 1-cyclopropyl-6-fluoro-1,4-dihydro-8-methoxy-7-(3-methyl-1-piperazinyl)-4-oxo-3-quinolinecarboxylic acid). Gatifloxacin hemihydrate has the chemical formula C₁₉H₂₂FN₃O₄.½ H₂O. Gatifloxacin sesquihydrate has the chemical formula C₁₉H₂₂FN₃O₄.1.5 H₂O. Gatifloxacin is administered as a racemate, with the disposition and antibacterial activity of the R- and S-enantiomers virtually identical. Gatifloxacin is used as an antibacterial.

Gatifloxacin is commercially-available as TEQUIN® Tablets from Bristol-Meyers Squibb Company. TEQUIN® Tablets are available as 200 mg and 400 mg of gatifloxacin sesquihydrate, and contain the following inactive ingredients: hydroxypropyl methylcellulose, magnesium stearate, methylcellulose, microcrystalline cellulose, polyethylene glycol, polysorbate 80, simethicone, sodium starch glycolate, sorbic acid and titanium dioxide.

U.S. Pat. No. 5,043,450 describes quinolonecarboxylic acid derivatives, such as ofloxacin and ciprofloxacin.

U.S. Pat. No.6,291,462 describes tablets having a multi-phase structure consisting of an inner phase and at least one outer phase, said inner phase comprising a gatifloxacin compound selected from the group consisting of gatifloxacin, pharmaceutically acceptable salts thereof and hydrates thereof, and at least one auxiliary substance selected from the group consisting of fillers, binders and disintegration aids; and said outer phase comprising at least one disintegration aid and at least one lubricant. The tablets are prepared by a wet granulation process using 20-80 weight percent (wt. %) of a granulating liquid, wherein the weight percents are based on the total amount of inner phase components.

### SUMMARY OF THE INVENTION

The invention provides a method for preparing a stable gatifloxacin composition in oral dosage form, said method comprising:

(a) mixing gatifloxacin or a pharmaceutically acceptable salt or hydrate thereof, a disintegrant and a filler for a time sufficient to form a mixture;

(b) compacting the mixture of Step (a) at a compaction force of about 1 kiloNewtons (kN) to about 30 kN for a time sufficient to form a compact;

(c) milling the compact of Step (b) to form a granulation; and

(d) mixing the granulation of Step (c) with an effective amount of a lubricant for a time sufficient to produce a gatifloxacin composition having a tap density of at least about 0.50 g/milliliters (mL).

According to another aspect, the invention provides a method for treating a bacterial infection comprising administering to a mammal in need of such treatment, a stable gatifloxacin composition in oral dosage form, wherein said composition is prepared by a method comprising:

(a) mixing gatifloxacin or a pharmaceutically acceptable salt or hydrate thereof, a disintegrant and a filler for a time sufficient to form a mixture;

(b) compacting the mixture of Step (a) at a compaction force of about 1 kN to about 30 kN for a time sufficient to form a compact;

(c) milling the compact of Step (b) to form a granulation; and

(d) mixing the granulation of Step (c) with an effective amount of a lubricant for a time sufficient to produce a gatifloxacin composition having a tap density of at least about 0.50 g/mL.

The compositions of the invention surprisingly have substantially uniform physical and chemical characteristics. As used herein, “stable” means that the gatifloxacin in the compacted gatifloxacin composition is substantially free of other polymorphic forms of gatifloxacin, i.e., there are no signs of polymorphic change in gatifloxacin prepared according to the method of the invention, as determined by differential scanning calorimetry. This result is particularly surprising in view of the large amount of heat generated during the compaction step which could normally produce polymorphic forms.

### DESCRIPTION OF THE INVENTION

The present invention relates to a method for preparing a stable gatifloxacin composition for oral dosage form. In the first step, Step (a), gatifloxacin or a pharmaceutically acceptable salt or hydrate thereof, is mixed with a disintegrant and a filler for a time sufficient to form a mixture or a blend.

Gatifloxacin is 1-cyclopropyl-6-fluoro-1,4-dihydro-8-methoxy-7-(3-methyl-1-piperazinyl)-4-oxo-3-quinolinecarboxylic acid). Gatifloxacin hemihydrate has the chemical formula C₁₉H₂₂FN₃O₄.½ H₂O. Gatifloxacin sesquihydrate has the chemical formula C₁₉H₂₂FN₃O₄.1.5 H₂O. Salts of gatifloxacin include, e.g., those with inorganic acids, such as hydrochloric acid, sulfuric acid, phosphoric acid, etc.; those with organic acids, such as methanesulfonic acid, lactic acid, oxalic acid, acetic acid, etc.; or salts of sodium, potassium, magnesium, calcium, aluminum, cerium, chromium, cobalt, copper, iron, zinc, platinum, silver, etc.

The gatifloxacin or a pharmaceutically acceptable salt or hydrate thereof, is preferably present in the compositions of the invention in an amount of from about 10 wt. % to about 95 wt. %, based on the total weight of the composition. More preferably, the gatifloxacin or a pharmaceutically acceptable salt or hydrate thereof is present in an amount of from about 40 wt. % to about 85 wt. %, most preferably, from about 50 wt. % to about 75 wt. %, based on the total weight of the composition.

Suitable disintegrants include pharmaceutically acceptable disintegrants which are chemically and physically compatible with gatifloxacin or a pharmaceutically acceptable salt or hydrate thereof. Preferably, the disintegrant is selected from the following: croscarmellose sodium, sodium starch glycolate, pregelatinized starches, sodium carboxymethyl cellulose, microcrystalline cellulose, magnesium aluminium silicate, bentonite, alginic acid and alginates. A mixture of disintegrants may also be used. More preferably, the disintegrant is sodium starch glycolate.

An effective amount of a disintegrant useful in the compositions of the invention is preferably in the range of about 1 wt. % to about 15 wt. %, more preferably about 3 wt. % to about 10 wt. %, and most preferably about 6 wt. % to about 8 wt. %, based on the total weight of the composition.

Suitable fillers include any such pharmaceutically acceptable filler which gives the powder composition bulk and which is physically and chemically compatible with gatifloxacin or a pharmaceutically acceptable salt or hydrate thereof. Preferably, the filler is selected from the following: lactose anhydrous, lactose monohydrate, sucrose, mannitol, microcrystalline cellulose, pre-gelatinized starches, dibasic calcium phosphate dihydrate, calcium sulfate trihydrate and calcium sulfate dihydrate. A mixture of fillers may also be used. More preferably, the filler is microcrystalline cellulose.

An effective amount of a filler useful in the compositions of the invention is preferably in the range of about 1 wt. % to about 50 wt. %, preferably about 15 wt. % to about 40 wt. %, and most preferably about 20 wt. % to about 30 wt. % of the composition.

In one embodiment of the invention, a lubricant is added to the mixture comprising gatifloxacin or a pharmaceutically acceptable salt or hydrate thereof, a disintegrant and a filler. Suitable lubricants include any pharmaceutically acceptable solid or liquid lubricants which are used to enhance the flow and prevent sticking of the composition after compaction and which are chemically and physically compatible with gatifloxacin or a pharmaceutically acceptable salt or hydrate thereof. Preferably, the lubricant is selected from the following: magnesium stearate, calcium stearate, zinc stearate, talc and stearic acid. A mixture of lubricants may also be used. More preferably, the lubricant is magnesium stearate.

An effective amount of a lubricant useful in the compositions of the invention is preferably in the range of about 0.1 wt. % to about 5 wt. %, more preferably about 0.5 wt. % to about 3 wt. %, and most preferably about 1 wt. % to about 2 wt. %, based on the total weight of the composition.

In the second step, Step (b), the mixture of Step (a) is passed through a roller compactor at a compaction force in the range of about 1-30 kN for a time sufficient to produce a compact. Preferably, the compaction force in the range of about 10-25 kN, more preferably about 20 kN. Preferably, the compact that is in the form of a gatifloxacin or a pharmaceutically acceptable salt or hydrate thereof, which is homogeneous, and substantially free, i.e., more than 95% free, of other polymorphic forms of gatifloxacin. In one embodiment of the invention, a Bepex roller compactor is used as the roller compactor. In addition to the compaction force, another suitable process parameters, in particular, for a Bepex roller compactor is preferably a roller speed of about 5 revolutions per minute (rpm).

In the third step, Step (c), the compact of Step (b) is milled to form a granulation. Types of mills which may be used in the invention include, but are not limited to, hammer mill, cutting mill, co-mill, and oscillating granulator. More specifically, suitable mills include, Quadro, Fitzpatrick (Fitz mill), BTS mill and Tornado. A preferred mill is a Fitz mill.

In the fourth step, Step (d), the granulation of Step (c) is mixed with an effective amount of a lubricant for a time sufficient to produce a gatifloxacin composition preferably having a homogeneous distribution of gatifloxacin or a pharmaceutically acceptable salt or hydrate thereof, preferably exhibiting a blend uniformity of about 90% to about 110%; a relative standard deviation (RSD) of less than about 5%; and a tap density of at least about 0.50 g/mL. Preferably, the gatifloxacin composition has a tap density of about 0.60 g/mL to about 0.85 g/mL. Most preferably, the tap density of the compositions is about 0.75 g/mL to about 0.80 g/mL. The term “tap density”, as used herein, means the measured mass of a powder attained at a limiting volume measured in a cylinder after being “tapped down”, typically by a mechanical device; typically tap density is recorded as mass in grams divided by volume in mL. The tap density is measured in accordance with the procedure described in USP 23, NF 18, Supplement 6 (1997), procedure 616, at Page 3768.

Suitable lubricants include any pharmaceutically acceptable solid or liquid lubricants which are used to enhance the flow and prevent sticking of the composition after compaction and which are chemically and physically compatible with gatifloxacin or a pharmaceutically acceptable salt or hydrate thereof. Preferably, the lubricant is selected from the following: magnesium stearate, calcium stearate, zinc stearate, talc and stearic acid. A mixture of lubricants may also be used. More preferably, the lubricant is magnesium stearate.

An effective amount of a lubricant useful in the compositions of the invention is preferably in the range of about 0.1 wt. % to about 5 wt. %, more preferably about 0.5 wt. % to about 3 wt. %, and most preferably about 1 wt. % to about 2 wt. %, based on the total weight of the composition.

It is within the scope of the invention for the compositions of the invention to include one or more pharmaceutically acceptable excipients in addition to a disintegrant(s), filler(s) and lubricant(s). Examples of such excipients are stabilizers, anti-oxidants, anti-adherents, preservatives and glidants. A combination of excipients may also be used. Such excipients are known to those skilled in the art.

The compositions of the invention are preferably provided in oral dosage form, such as, tablets, granules, hard or soft capsules and powders. More preferably, the dosage form is a tablet. The term “tablet” includes uncoated tablets, coated tablets, matrix tablets, osmotic tablets and other forms known in the art. The oral dosage forms may be immediate-release, extended-release or controlled-release.

In one embodiment of the invention, the invention relates to a method for treating a bacterial infection comprising administering to a mammal in need of such treatment, including a human patient, a therapeutically effective amount of a stable gatifloxacin composition in oral dosage form, wherein said composition is prepared by a method comprising:

(a) mixing gatifloxacin or a pharmaceutically acceptable salt or hydrate thereof, a disintegrant and a filler for a time sufficient to form a mixture;

(b) compacting the mixture of Step (a) at a compaction force of about 10 kN to about 30 kN for a time sufficient to form a compact;

(c) milling the compact of Step (b) to form a granulation; and

(d) mixing the granulation of Step (c) with an effective amount of a lubricant for a time sufficient to produce a gatifloxacin composition having a tap density of at least about 0.50 g/mL.

The following non-limiting examples illustrate further aspects of the invention.

### EXAMPLE 1

Polymorphic Conversion Testing after Compression at 1 Ton Pressure and Storage in Different Environments.

Compacts of glatifloxacin anhydrous and gatifloxacin hemihydrate were prepared and placed in chambers that were equilibrated as follows:

(i) 25° C. and 21% relative humidity (RH);

(ii) 25° C. and 75% RH;

(iii) 40° C. and 21% RH; and

(iv) 40° C. and 21% RH.

Compacts of glatifloxacin anhydrous and gatifloxacin hemihydrate were evaluated by DSC and FT-IR immediately after compression, after 2 days of exposure under the above conditions, and after 6 or 7 days of exposure.

Conversion is identified as a change in DSC thermogram or FT-IR pattern. The test results are summarized in Table I.

TABLE I
Effect of Compression and Storage on Physical Stability
of Gatifloxacin Anhydrous and Gatifloxacin Hemihydrate.

| | Gatifloxacin | Gatifloxacin |
|---|---|---|
| | Anhydrous | Hemihydrate |
| | DSC/FT-IR | DSC/FT-IR |
| Immediately after compression | No/No | No/No |
| 2 Days at 25° C. and 21% RH | No/No | No/No |
| 2 Days at 25° C. and 75% RH | No/No | No/No |
| 2 Days at 40° C. and 21% RH | No/No | No/No |
| 2 Days at 40° C. and 75% RH | Yes/Yes | No/No |
| 6 or 7 Days at 25° C. and 21% RH | No/No | No/No |
| 6 or 7 Days at 25° C. and 75% RH | Yes/Yes | Yes/No |
| 6 or 7 Days at 40° C. and 21% RH | No/No | No/No |
| 6 or 7 Days at 40° C. and 75% RH | Yes/Yes | Yes/Yes |

The results in Table I show that a higher RH has more of an effect than a higher temperature on the physical stability of glatifloxacin anhydrous and glatifloxacin hemihydrate. For example, RH conditions of approximately 75% RH for a period of 2 days deleteriously effects the physical stability of glatifloxacin anhydrous. However, 6 or 7 days at 40° C. and 21% RH did not effect the glatifloxacin anhydrous. In addition, RH conditions of approximately 75% RH for a period of at least 6 days deleteriously effects the physical stability of glatifloxacin hemihydrate. However, 6 or 7 days at 40° C. and 21% RH did not effect the glatifloxacin hemihydrate.

### EXAMPLE 2

Preparation of a Gatifloxacin Tablets (400 mg).

| Ingredient | Amount | |
|---|---|---|
| Gatifloxacin Hemihydrate | 409.59 | mg |
| Magnesium Stearate, NF | 4.1 | mg |
| Microcrystalline Cellulose 200, NF | 156.31 | mg |
| Sodium Starch Glycolate, NF | 24.0 | mg |
| Magnesium Stearate, NF | 6.0 | mg |
| Total | 600 | mg |

Gatifloxacin hemihydrate, microcrystalline cellulose, sodium starch glycolate and magnesium stearate are mixed in a PMA high shear mixer for about 5 minutes to form a premix. The premix is passed through a Comminutor IR 520 (Fitzpatrick) roller compactor operated at a compaction force which is set forth in Table II for a time sufficient to produce three different compacts, denoted Compact A, Compact B and Compact C. Each of the compacts is passed through a Fitz-mill equipped with a 0.065″ perforated screen with knife forward setting at slow speed to form a granulation. Magnesium stearate is added to the granulation and mixed. The final mix blend is compressed using a 0.3400′×0.6400″ modified oval shape embossed tooling for the 400 mg tablets to provide three samples of uncoated tablets. The uncoated tablets are coated with a dispersion of Opadry White YS-1-7003 using coating equipment to a target of 3% solids weight gain.

TABLE II
Process Parameters Used for the Roller Compaction

| Process Parameter | Compact A | Compact B | Compact C |
|---|---|---|---|
| Roller Speed (rpm) | 10 | 10 | 10 |
| Vertical Feed | 200 | 200 | 200 |
| Screw Speed (rpm) | | | |
| Horizontal Feed | 15 | 15 | 15 |
| Screw Speed (rpm) | | | |
| Roller Pressure (psi) | 600 | 800 | 1000 |

### EXAMPLE 3

Dissolution Profile of Tablets Prepared in Example 2.

The disintegration of tablets is a test method for providing evidence about a well-defined medicinal form. To determine the disintegration, tablets are placed in an apparatus, the main part of which generally consists of a rigid frame with a perforated bottom containing, e.g., 6 cylindrical glass test tubes of fixed dimensions. Each tube can be fitted with a disc of a translucent plastic material or comparable materials which have specific orifices and V-shaped indentations. The test tubes are held vertical by an upper and a lower plate which can be made of plastic. On the underside there is a stainless steel wire gauze with a mesh size of 2 mm. The apparatus is moved uniformly up and down 28-32 times a minute by means of a motor. The apparatus is suspended in a vessel containing water. The amount of water present in the vessel is such that the wire gauze is still immersed below the surface of the water at the highest point of its travel and is still above the bottom of the vessel at its lowest point, and the mouths of the tubes remain above the surface of the water. The water is kept at a temperature of 36-38° C.

The dissolution results are summarized in Table III.

TABLE III

| Time | % Drug Dissolved | % Drug Dissolved | % Drug Dissolved |
|---|---|---|---|
| (min.) | Sample A | Sample B | Sample C |
| 5 | 45.4% | 63.1% | 64.8% |
| 10 | 52.7% | 70.1% | 71.3% |
| 15 | 56.3% | 72.9% | 74.4% |
| 20 | 59.2% | 76.0% | 75.8% |
| 30 | 64.6% | 76.6% | 78.5% |
| 45 | 72.5% | 80.3% | 81.1% |
| 60 | 80.1% | 82.4% | 83.3% |
| 75 | 88.1% | 98.9% | 99.7% |

The results in Table III show that at least 80% of gatifloxacin in the compositions prepared according to the invention dissolved in water in about 60 minutes.

### EXAMPLE 4

Preparation of a Gatifloxacin Tablets (200 mg) by Roller Compaction.

| Ingredient | Amount | |
|---|---|---|
| Pre-mix I | | |
| Gatifloxacin Hemihydrate | 204.8 | mg |
| Microcrystalline Cellulose 102, NF | 76.2 | mg |
| Sodium Starch Glycolate, NF | 24.0 | mg |
| Pre-mix II | | |
| Magnesium Stearate, NF | 2.0 | mg |
| Final-mix | | |
| Magnesium Stearate, NF | 3.0 | mg |
| Total | 310.0 | mg |
| Coated Tablets | | |
| Opadry Yellow YS-1-2053 | 9.0 | mg |
| Purified Water, USP | q.s. | |
| Total | 319.0 | mg |

Gatifloxacin hemihydrate, microcrystalline cellulose, and sodium starch glycolate are mixed in a 300 liter (L) Fielder high-shear mixer for about 5 minutes to form Premix I. Magnesium stearate is added to Premix I and mixed for about 1 minute to form Premix II. Premix II is passed through a Bepex roller compactor operated at a compaction force of 20 kN and a roller speed of 5 rpm. The resulting compact is passed through a Fitz-mill equipped with a 0.065″ perforated screen with knife forward setting at slow speed to form granules. Magnesium stearate is added to the granules and mixed to form a Final mix. The Final mix is compressed in a rotary tablet press using a 0.2700″×0.5080″ modified oval shape embossed tool to form uncoated tablets. The uncoated tablets are coated with a dispersion of Opadry Yellow YS-1-2053 using an Accela Coater perforated coating pan to a target of 3% solids weight gain.

### EXAMPLE 5

Preparation of a Gatifloxacin Tablets (400 mg) by Roller Compaction.

| Ingredient | Amount | |
|---|---|---|
| Pre-mix I | | |
| Gatifloxacin Hemihydrate | 409.6 | mg |
| Microcrystalline Cellulose 102, NF | 152.4 | mg |
| Sodium Starch Glycolate, NF | 48.0 | mg |
| Pre-mix II | | |
| Magnesium Stearate, NF | 4.0 | mg |
| Final-mix | | |
| Magnesium Stearate, NF | 6.0 | mg |
| Total | 620.0 | mg |
| Coated Tablets | | |
| Opadry Yellow YS-1-2053 | 18.0 | mg |
| Purified Water, USP | q.s. | |
| Total | 638.0 | mg |

Gatifloxacin hemihydrate, microcrystalline cellulose, sodium and starch glycolate are mixed in a 300 L Fielder high-shear mixer for about 5 minutes to form Premix I. Magnesium stearate is added to Premix I and mixed for about 1 minute to form Premix II. Premix II is passed through a Bepex roller compactor operated at a compaction force of 20 kN and a roller speed of 5 rpm. The resulting mix is passed through a Fitz-mill equipped with a 0.065″ perforated screen with knife forward setting at slow speed to form granules. Magnesium stearate is added to the granules and mixed to form a Final mix. The Final mix is compressed in a rotary tablet press using a 0.3400″×0.6400″ modified oval shape embossed tool to form uncoated tablets. The uncoated tablets are coated with a dispersion of Opadry Yellow YS-1-2053 using an Accela Coater perforated coating pan to a target of 3% solids weight gain.

While the invention has been described with particular reference to certain embodiments thereof, it will be understood that changes and modifications may be made by those of ordinary skill within the scope and spirit of the following claims:

What is claimed is:

1. A method for preparing a stable gatifloxacin composition in oral dosage form, said method comprising:
(a) mixing gatifloxacin or a pharmaceutically acceptable salt or hydrate thereof, a disintegrant, and a filler for a time sufficient to form a mixture;
(b) compacting the mixture of Step (a) at a compaction force of about 1 kN to about 30 kN for a time sufficient to form a compact;
(c) milling the compact of Step (b) to form a granulation; and
(d) mixing the granulation of Step (c) with an effective amount of a lubricant for a time sufficient to produce a gatifloxacin composition having a tap density of at least about 0.50 g/mL.

2. The method according to claim 1, wherein the gatifloxacin or a pharmaceutically acceptable salt or hydrate thereof is gatifloxacin hemihydrate.

3. The method according to claim 1, wherein the gatifloxacin or a pharmaceutically acceptable salt or hydrate thereof is present in an amount of from about 10 weight percent (wt. %) to about 95 wt. %, based on the total weight of the composition.

4. The method according to claim 3, wherein the gatifloxacin or a pharmaceutically acceptable salt or hydrate thereof is present in an amount of from about 50 wt. % to about 75 wt. %, based on the total weight of the composition.

5. The method according to claim 1, wherein the disintegrant is present in an amount of from about 1 wt. % to about 15 wt. %, the filler is present in an amount of from about 1 wt. % to about 50 wt. % and the lubricant is present in an amount of from about 0.1 wt. % to about 5 wt. %, based on the total weight of the composition.

6. The method according to claim 5, wherein the disintegrant is present in an amount of from about 3 wt. % to about 10 wt. %, the filler is present in an amount of from about 15 wt. % to about 40 wt. %, and the lubricant is present in an amount of from about 0.5 wt. % to about 3 wt. %, based on the total weight of the composition.

7. The method according to claim 6, wherein the disintegrant is present in an amount of from about 6 wt. % to about 8 wt. %, the filler is present in an amount of from about 20 wt. % to about 30 wt. %, and the lubricant is present in an amount of from about 1 wt. % to about 2 wt. %, based on the total weight of the composition.

8. The method according to claim 1, which additionally comprises adding a lubricant prior to compacting the mixture in Step (b).

9. The method according to claim 1, wherein the disintegrant is selected from the group consisting of croscarmellose sodium, sodium starch glycolate, pregelatinized starches, sodium carboxymethyl cellulose, microcrystalline cellulose, magnesium aluminium silicate, bentonite, alginic acid, alginates and mixtures thereof.

10. The method according to claim 9, wherein the disintegrant is sodium starch glycolate.

11. The method according to claim 1, wherein the filler is selected from the group consisting of lactose anhydrous, lactose monohydrate, sucrose, mannitol, microcrystalline cellulose, pregelatinized starches, dibasic calcium phosphate dihydrate, calcium sulfate trihydrate, calcium sulfate dihydrate and mixtures thereof.

12. The method according to claim 11, wherein the filler is microcrystalline cellulose.

13. The method according to claim 1, wherein the lubricant is selected from the group consisting of magnesium stearate, calcium stearate, zinc stearate, talc, stearic acid and mixtures thereof.

14. The method according to claim 13, wherein the lubricant is magnesium stearate.

15. The method according to claim 1, wherein the compaction force in Step (b) is from about 10 kN to about 25 kN.

16. The method according to claim 15, wherein the compression force in Step (b) is about 20 kN.

17. The method according to claim 1, wherein the composition has a tap density of about 0.60 g/mL to about 0.85 g/mL.

18. The method according to claim 17, wherein the composition has a tap density of about 0.75 g/mL to about 0.80 g/mL.

19. A stable gatifloxacin composition in the form of a tablet, wherein said composition is prepared by a method comprising:
(a) mixing gatifloxacin or a pharmaceutically acceptable salt or hydrate thereof, a disintegrant and a filler for a time sufficient to form a mixture;
(b) compacting the mixture of Step (a) at a compaction force of about 1 kN to about 30 kN for a time sufficient to form a compact;
(c) milling the compact of Step (b) to form a granulation; and
(d) mixing the granulation of Step (c) with an effective amount of a lubricant for a time sufficient to produce a gatifloxacin composition having a tap density of at least about 0.50 g/mL.

20. A method for treating a bacterial infection comprising administering to a mammal in need of such treatment, a stable gatifloxacin composition in oral dosage form, wherein said composition is prepared by a method comprising:
(a) mixing gatifloxacin or a pharmaceutically acceptable salt or hydrate thereof, a disintegrant and a filler for a time sufficient to form a mixture;
(b) compacting the mixture of Step (a) at a compaction force of about 1 kN to about 30 kN for a time sufficient to form a compact;
(c) milling the compact of Step (b) to form a granulation; and
(d) mixing the granulation of Step (c) with an effective amount of a lubricant for a time sufficient to produce a gatifloxacin composition having a tap density of at least about 0.50 g/mL.
